(19) United States
(12) Patent Application Publication
(10) Pub. No.: US 2019/0387697 A1
(43) Pub. Date: Dec. 26, 2019

(54) METHOD OF PRODUCING PATHOGEN-FREE CANNABIS PLANTS AND PATHOGEN-FREE PLANTS AND CLONES PRODUCED THEREFROM

(71) Applicant: Grace Executive Services, Inc., Oakland, CA (US)

(72) Inventors: Daniel J. GRACE, San Leandro, CA (US); William J. ROBERTS, Oakland, CA (US)

(73) Assignee: Grace Executive Services, Inc., Oakland, CA (US)

(21) Appl. No.: 16/013,787

(22) Filed: Jun. 20, 2018

(51) Int. Cl. A01H 4/00; A01H 6/28; A01H 5/02; A01H 3/04

(52) U.S. Cl. CPC A01H 4/001; A01H 6/28; A01H 5/02; A01N 43/90; A01H 4/005; A01H 3/04; A01H 4/008

(57) ABSTRACT

Disclosed herein are methods of producing substantially pathogen-free plants of the genus Cannabis and pathogen-free plants and clones produced therefrom. One embodiment of the method comprises heating a progenitor plant of the genus Cannabis within a heating chamber resulting in a heat-treated plant, surface sterilizing a shoot segment of the heat-treated plant with a bleach solution, excising a meristematic tip of the shoot segment, and transferring the meristematic tip into a culturing plate comprising a supplemented Murashige and Skoog culture medium for further culturing. The supplemented Murashige and Skoog culture medium can comprise benzyladenine, naphthaleneacetic acid, gibberellic acid, or a combination thereof.

### TECHNICAL FIELD

This disclosure relates generally to the field of plant husbandry and cultivation, more specifically, to methods of producing substantially pathogen-free plants of the genus Cannabis and substantially pathogen-free plants and clones produced from such methods.

### BACKGROUND

The legalization of cannabis for recreational and medicinal use in states such as Colorado, Washington, Oregon, Alaska, and California has resulted in a deluge of growers entering the cannabis cultivation space. However, the rise in cannabis plant stock as a whole has also brought along an attendant rise in pathogen infection rates among such plant populations. While viruses such as Tobacco Mosaic Virus (TMV), Cannabis Cryptic Virus (CCV), and Hop Mosaic Virus (HpMV) have been known to infect plants of the genus Cannabis, a recently discovered pathogen has begun to raise alarms in numerous grow-houses, nurseries, and farms. Those in the cannabis industry refer to the new pathogen as Putative Cannabis Infectious Agent or PCIA and plants infected with PCIA are often referred to as “duds.”

FIG. 1 is a black-and-white image of a side-by-side comparison of a healthy Cannabis sativa plant on the right side and a Cannabis sativa plant infected by PCIA on the left. As shown in FIG. 1, the infected plant exhibits a lack of apical dominance (i.e., the side branches grow more than the central stem), diminished leaf size, bowed braches or excessive branching, increased internode spacing, and brittle stems. Infected plants also often do not produce flowers that mature and do not produce the type of resins or oils typical of healthy cannabis plants.

Plant epidemiological studies conducted of farms in the Humboldt, Calif. region in 2015 revealed that up to 20% to 35% of plants in the farms surveyed were infected with PCIA. PCIA has been known to spread by plant-on-plant contact, insect vectors, and human or tool contact with infected plants. The ease and speed by which PCIA can spread in both contained and outdoor plant environments have made the pathogen a prime concern for the cannabis industry.

While PCIA has been attributed to one or more viruses or viroids, studies are still ongoing to understand the pathogen. The uncertainty concerning PCIA has opened the door for many unproven treatment methods to take hold within the cannabis industry. Such treatment methods include immersing diseased plants or plant parts in alcohol, continuously replacing the soil of diseased plants, and the excessive use of harmful insecticides. However, such methods have not been shown to reliably reduce the incidence of PCIA or viral infections in treated plant populations. Moreover, such methods may worsen the problem when diseased plants treated by such ineffective methods are introduced back into the nursery stock.

Therefore, improved methods of producing substantially pathogen-free plants of the genus Cannabis are needed. In addition, such methods should be cost-effective and easy to implement on a large-scale. Moreover, such a method should result in plant populations that exhibit, in general, a lower rate of pathogen infections and are robust and healthy.

### SUMMARY

Methods of producing substantially pathogen-free plants of the genus Cannabis and substantially pathogen-free plants and clones produced from such methods are disclosed herein. One embodiment of the method can comprise heating a progenitor plant of the genus Cannabis within a heating chamber resulting in a heat treated plant. The progenitor plant can be in a vegetative growth stage when heated. Heating the progenitor plant can comprise heating the progenitor plant at alternating temperatures of approximately 100° F. and 85° F. The progenitor plant can be heated at each temperature for approximately a four hour period for a total of 14 days. The progenitor plant can be between approximately 6 inches and 18 inches in height, as measured from the soil surface, when subjected to heat-treatment.

The method can further comprise surface sterilizing a shoot segment of the heat-treated plant with a bleach solution. Surface sterilizing the shoot segment of the heat-treated plant can comprise immersing the shoot segment in the bleach solution for between approximately 10 minutes and 20 minutes or, more specifically, 15 minutes. The bleach solution can comprise approximately 2.475% (w/v %) of sodium hypochlorite.

The method can also comprise excising a meristematic tip of the shoot segment. Excising the meristematic tip of the shoot segment can comprise excising an apical portion of the shoot segment equal to or less than approximately 0.5 mm in size. The apical portion of the shoot segment can comprise meristem tissue. The meristematic tip can be excised using a scalpel under microscopy.

The method can further comprise transferring the meristematic tip into a culturing plate comprising a supplemented Murashige and Skoog culture medium for further culturing. The supplemented Murashige and Skoog culture medium can comprise benzyladenine (6-benzylaminopurine), naphthaleneacetic acid, and gibberellic acid. In one embodiment, the supplemented Murashige and Skoog culture medium can comprise 1.0 mg/L of benzyladenine (6-benzylaminopurine), 0.1 mg/L of naphthaleneacetic acid, and 0.1 mg/L of gibberellic acid.

The method can further comprise transferring a plantlet grown from the meristematic tip from the culturing plate into a test tube comprising additional supplemented Murashige and Skoog culture medium after approximately 21 days to 30 days. The method can also comprise transferring the plantlet growing in the test tube from the test tube into a large-tissue culture vessel comprising Murashige and Skoog culture medium after approximately 28 days to 56 days.

The method can further comprise transferring the plantlet growing in the large-tissue culture vessel into a first rooting medium after 28 days to 56 days to yield a young elite mother plant. In addition, the method can comprise transferring the young elite mother plant and at least a portion of the first rooting medium into a second rooting medium after approximately 10 days to 16 days and growing the young elite mother plant in the second rooting medium between 7 days and 28 days to yield an elite mother plant.

A plant of the genus Cannabis is also disclosed. The plant is produced by a process comprising the steps of heating a progenitor plant of the genus Cannabis within a heating chamber resulting in a heat treated plant. The progenitor plant can be in a vegetative growth stage when heated. Heating the progenitor plant can comprise heating the progenitor plant at alternating temperatures of approximately 100° F. and 85° F. The progenitor plant can be heated at each temperature for approximately a four hour period for a total of 14 days. A height dimension of the progenitor plant heated in the heating chamber can be between approximately 6 inches and 18 inches, as measured from the soil surface.

The process can further comprise surface sterilizing a shoot segment of the heat-treated plant with a bleach solution. Surface sterilizing the shoot segment of the heat-treated plant can comprise immersing the shoot segment in the bleach solution for between approximately 10 minutes and 20 minutes or, more specifically, 15 minutes. The bleach solution can comprise approximately 2.475% (w/v %) of sodium hypochlorite.

The process can also comprise excising a meristematic tip of the shoot segment. Excising the meristematic tip of the shoot segment can comprise excising an apical portion of the shoot segment equal to or less than approximately 0.5 mm in size. The apical portion of the shoot segment can comprise meristem tissue. The meristematic tip can be excised using a scalpel under microscopy.

The process can further comprise transferring the meristematic tip into a culturing plate comprising a supplemented Murashige and Skoog culture medium for further culturing. The supplemented Murashige and Skoog culture medium can comprise benzyladenine (6-benzylaminopurine), naphthaleneacetic acid, and gibberellic acid. In one embodiment, the supplemented Murashige and Skoog culture medium can comprise 1.0 mg/L of benzyladenine (6-benzylaminopurine), 0.1 mg/L of naphthaleneacetic acid, and 0.1 mg/L of gibberellic acid.

The process can further comprise transferring a plantlet grown from the meristematic tip from the culturing plate into a test tube comprising additional supplemented Murashige and Skoog culture medium after approximately 21 days to 30 days. The process can also comprise transferring the plantlet from the test tube into a large-tissue culture vessel comprising Murashige and Skoog culture medium after approximately 28 days to 56 days.

The process can further comprise transferring the plantlet growing in the large-tissue culture vessel into a first rooting medium after 28 days to 56 days to yield a young elite mother plant. In addition, the process can comprise transferring the young elite mother plant and at least a portion of the first rooting medium into a second rooting medium after 10 days to 16 days and growing the young elite mother plant in the second rooting medium between 7 days and 28 days to yield an elite mother plant.

A cloned plant of the genus Cannabis is also disclosed. The cloned plant is produced by a process comprising the steps of heating a progenitor plant of the genus Cannabis within a heating chamber resulting in a heat treated progenitor plant. The progenitor plant can be in a vegetative growth stage when heated. Heating the progenitor plant can comprise heating the progenitor plant at alternating temperatures of approximately 100° F. and 85° F. The progenitor plant can be heated at each temperature for approximately a four hour period for a total of 14 days. A height dimension of the progenitor plant heated in the heating chamber can be between approximately 6 inches and 18 inches, as measured from the soil surface.

The process can further comprise surface sterilizing a shoot segment of the heat-treated progenitor plant with a bleach solution. Surface sterilizing the shoot segment of the heat-treated plant can comprise immersing the shoot segment in the bleach solution for between approximately 10 minutes and 20 minutes or, more specifically, 15 minutes. The bleach solution can comprise approximately 2.475% (w/v %) of sodium hypochlorite.

The process can also comprise excising a meristematic tip of the shoot segment. Excising the meristematic tip of the shoot segment can comprise excising an apical portion of the shoot segment equal to or less than approximately 0.5 mm in size. The apical portion of the shoot segment can comprise meristem tissue. The meristematic tip can be excised using a scalpel under microscopy.

The process can further comprise transferring the meristematic tip into a culturing plate comprising a supplemented Murashige and Skoog culture medium for further culturing. The supplemented Murashige and Skoog culture medium can comprise benzyladenine (6-benzylaminopurine), naphthaleneacetic acid, and gibberellic acid. In one embodiment, the supplemented Murashige and Skoog culture medium can comprise 1.0 mg/L of benzyladenine (6-benzylaminopurine), 0.1 mg/L of naphthaleneacetic acid, and 0.1 mg/L of gibberellic acid.

The process can further comprise transferring a plantlet grown from the meristematic tip from the culturing plate into a test tube comprising additional supplemented Murashige and Skoog culture medium after approximately 21 days to 30 days. The process can also comprise transferring the plantlet from the test tube into a large-tissue culture vessel comprising Murashige and Skoog culture medium after approximately 28 days to 56 days.

The process can further comprise transferring the plantlet growing in the large-tissue culture vessel into a first rooting medium after 28 days to 56 days to yield a young elite mother plant. In addition, the process can comprise transferring the young elite mother plant and at least a portion of the first rooting medium into a second rooting medium after 10 days to 16 days and growing the young elite mother plant in the second rooting medium between 7 days and 28 days to yield an elite mother plant.

The process can also comprise obtaining a stem cutting of the elite mother plant and immersing at least a segment of the stem cutting in a rooting hormone solution. The segment of the stem cutting can be immersed in the rooting hormone solution for between approximately 5 seconds and 10 seconds. In some embodiments, the rooting hormone solution can comprise indole-3-butyric acid and 1-napthaleneacetic acid as active ingredients.

The process can further comprise transferring the stem cutting into a temperature-controlled rooting medium and further cultivating the stem cutting in the temperature-controlled rooting medium until roots form to yield the cloned plant. The temperature-controlled rooting medium can be a rock-wool rooting medium and the temperature of the rock-wool rooting medium can be heated and maintained at approximately 80° F. In some embodiments, the rock-wool rooting medium can be heated by a heating system (e.g., a hydronic heating system) or heating mat.

### BRIEF DESCRIPTION OF THE DRAWINGS

FIG. 1 is a black-and-white image of a side-by-side comparison of a healthy plant of the genus Cannabis on the right and a plant infected by PCIA on the left.

FIG. 2A illustrates certain steps of an embodiment of a method for producing substantially pathogen-free plants of the genus Cannabis.

FIG. 2B illustrates certain other steps of the embodiment of the method for producing substantially pathogen-free plants of the genus Cannabis.

FIG. 3A is a black-and-white image of an embodiment of a temperature-controlled growth chamber heating one or more progenitor plants.

FIG. 3B illustrates an embodiment of an example heating schedule undertaken by the temperature-controlled growth chamber to heat the one or more progenitor plants.

FIG. 4 is a black-and-white image of heat-treated plants.

FIG. 5A is a black-and-white image of a shoot segment of a heat-treated plant.

FIG. 5B illustrates an embodiment of a technique of surface sterilizing the shoot segment.

FIG. 6 illustrates the anatomy of a distal portion of a shoot segment comprising a meristematic tip.

FIG. 7A is a black-and-white image of plantlets grown from meristematic tips on culturing plates comprising supplemented Murashige and Skoog culture medium.

FIG. 7B is a black-and white image of a plantlet growing within a test tube comprising supplemented Murashige and Skoog culture medium.

FIG. 8A is a black-and-white image of three types of culturing vessels used as part of the method of producing substantially pathogen-free plants including culturing plates, test tubes, and large tissue-culture vessels.

FIG. 8B is a black-and-white image of young elite mother plants grown from meristematic tips and acclimated for ambient ex-vitro growing conditions.

FIG. 9A is a black-and-white image of a miniature-sized elite mother plant for further propagation via cloning.

FIG. 9B is a black-and-white image of a regular-sized elite mother plant for further propagation via cloning.

FIG. 10 illustrates an embodiment of certain steps of a method for producing cloned plants from substantially pathogen-free elite mother plants.

FIG. 11A is a black-and-white image of a stem cutting obtained from an elite mother plant.

FIG. 11B is a black-and-white image of a segment of the stem cutting immersed in a rooting hormone solution.

FIG. 12A is a black-and-white image of the stem cutting in a rooting medium.

FIG. 12B is a black-and-white image showing the stem cutting being further cultivated in a temperature-controlled rooting medium to yield a cloned plant.

FIG. 13 is a table showing the results of tests conducted on plants of the genus Cannabis produced by the methods described herein for incidence of PCIA infections.

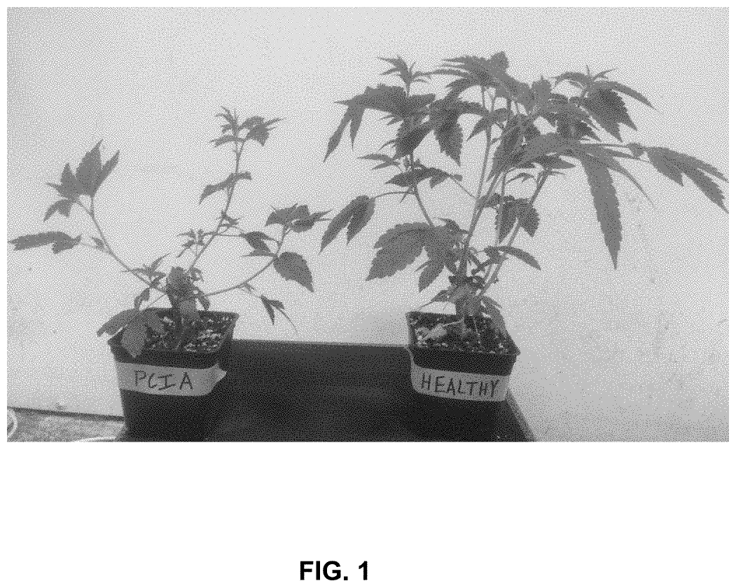

### DETAILED DESCRIPTION

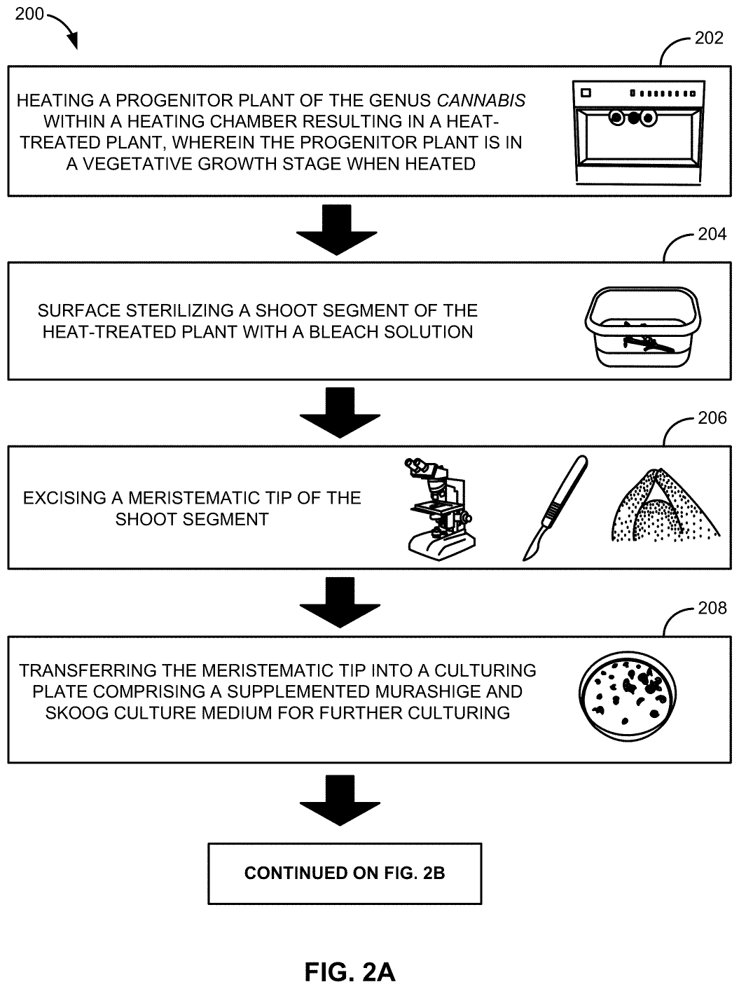

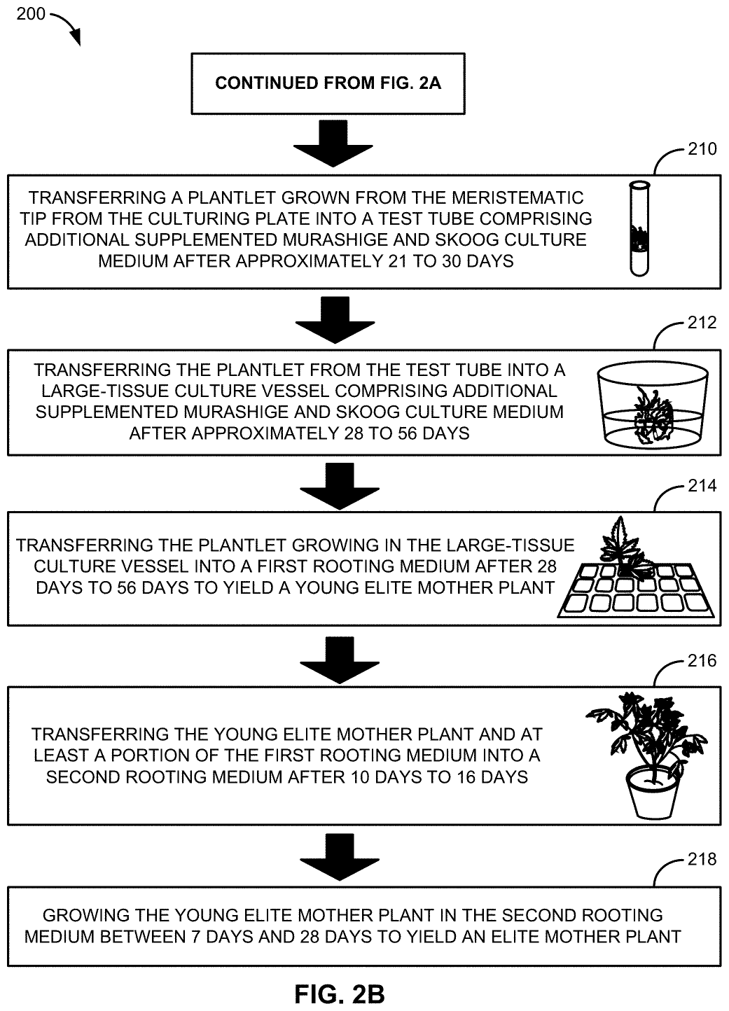

FIG. 2A illustrates certain steps of an embodiment of a method 200 for producing substantially pathogen-free plants of the genus Cannabis. Embodiments of the method 200 described herein can be applied to Cannabis sativa plants, Cannabis indica plants, or hybrids thereof.

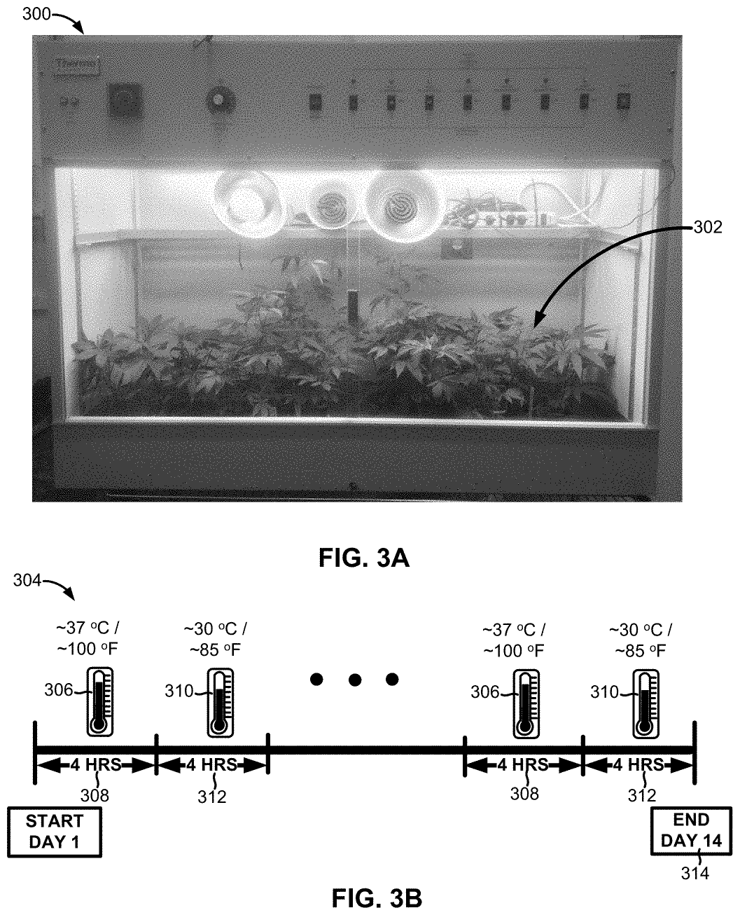

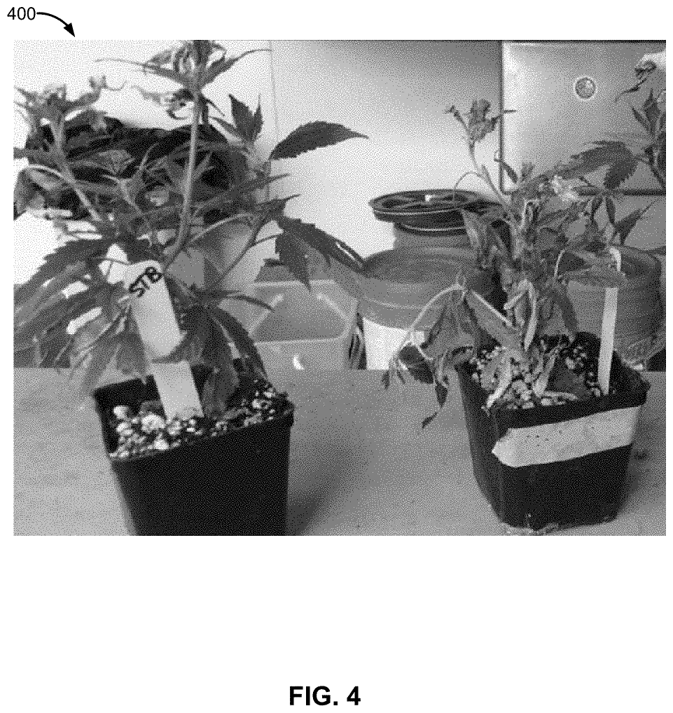

The method 200 can comprise heating a progenitor plant 302 (see FIG. 3A) of the genus Cannabis within a heating chamber 300 (see FIG. 3A) resulting in a heat-treated plant 400 (see FIG. 4) in step 202. The progenitor plant 302 can be in a vegetative growth stage when heated. In certain embodiments, the progenitor plant 302 can be between the ages of 2 weeks to 3 weeks when heated. In other embodiments, the progenitor plant 302 can be between the ages of 3 weeks to 4 weeks when heated. The progenitor plant 302 can be heated according to a heating schedule 304 (see FIG. 3B). The heating schedule 304 and the heating chamber 300 will be discussed in more detail in the following sections.

In some embodiments, the progenitor plant 302 can be a plant infected by a pathogen. In one embodiment, the pathogen can be PCIA. In other embodiments, the progenitor plant 302 can be a plant infected by another pathogen such as a virus from the family Virgaviridae, a virus from the family Betaflexiviridae, a viroid from the family Pospiviroidae, a viroid from the family Avsunviroidae, a phytoplasma, or a combination thereof. For example, the progenitor plant 302 can be a plant infected by a pathogen such as a virus from the genus Tobamovirus, a virus from the genus Carlavirus, a viroid from any of the genera Pospiviroid, Hostuviroid, Cocadviroid, Apscaviroid, or Coleviroid, a parasitic bacteria from the genus Candidatus Phytoplasma, or a combination thereof. In these and other embodiments, the progenitor plant 302 infected by the one or more pathogens can be exhibiting symptoms of infection or disease or be in an asymptomatic stage or phase. In further embodiments, the progenitor plant 302 can be a healthy plant of the genus Cannabis having been cultivated near or in proximity to other plants infected by the pathogen.

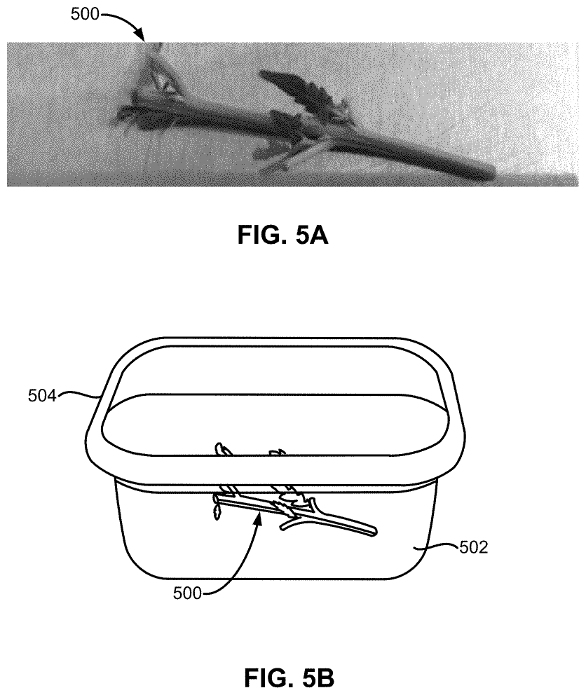

The method 200 can also comprise surface sterilizing a shoot segment 500 (see FIG. 5A) of the heat-treated plant 400 with a bleach solution 502 (see FIG. 5B) in step 204. Surface sterilizing the shoot segment 500 with the bleach solution 502 will be discussed in more detail in the following sections.

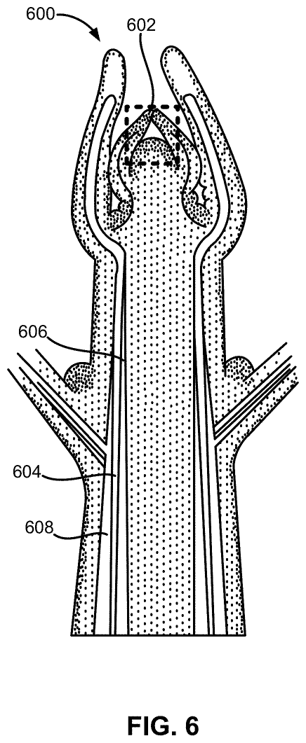

The method 200 can further comprise excising a meristematic tip 602 (see FIG. 6) of the sterilized instance of the shoot segment 500 in step 206. The meristematic tip 602 can be excised using a scalpel under microscopy. Excising the meristematic tip 602 will be discussed in more detail in the following sections.

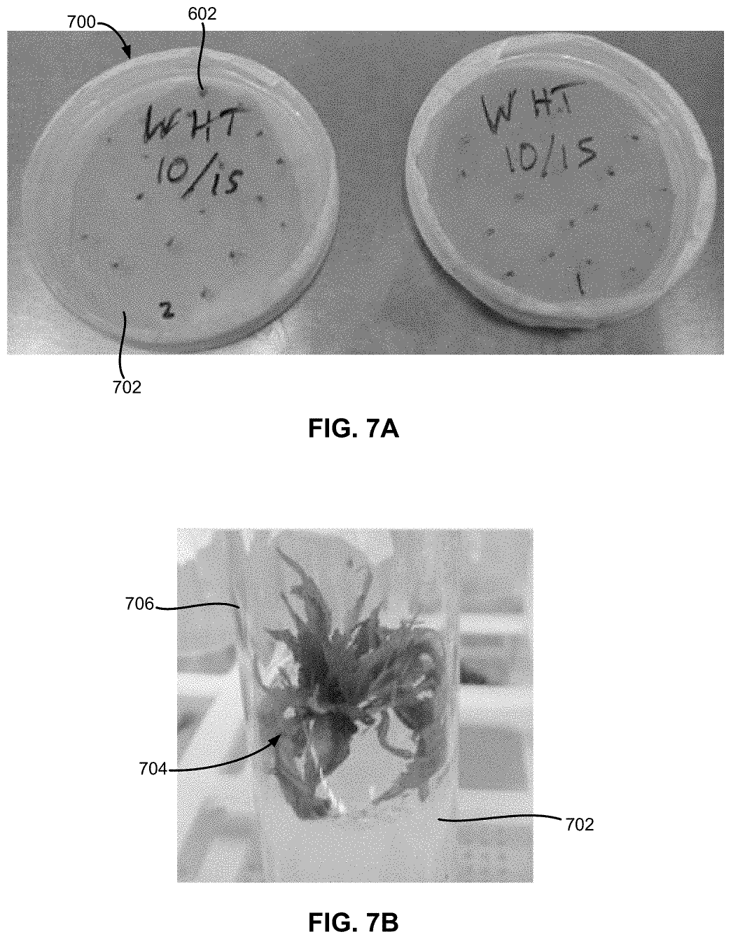

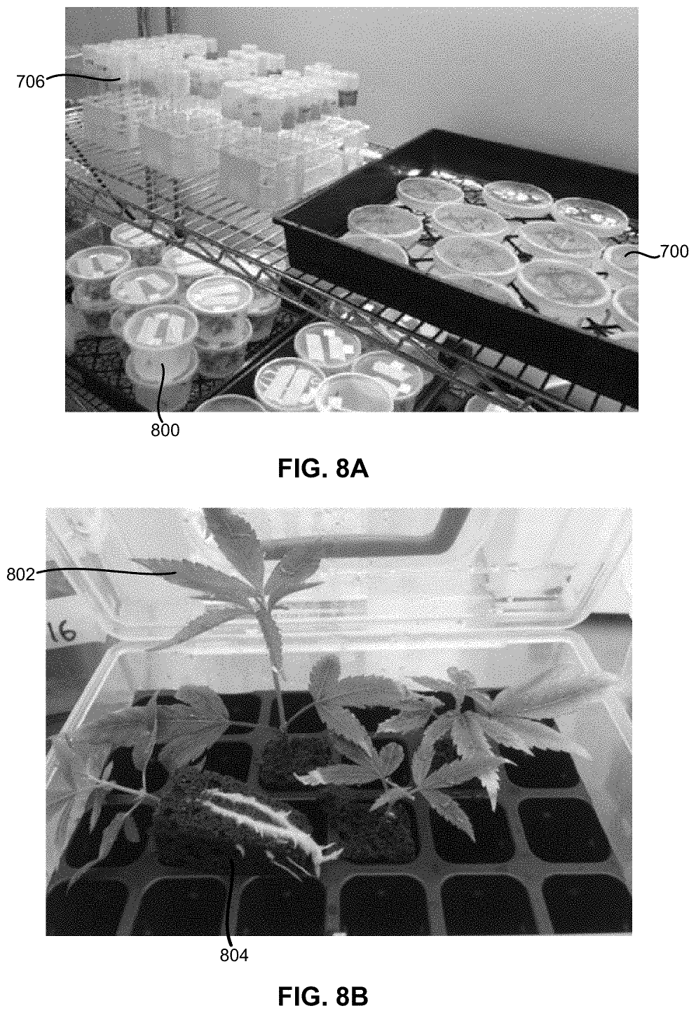

The method 200 can also comprise transferring the meristematic tip 602 into a culturing plate 700 (see FIGS. 7A and 8A) comprising a supplemented Murashige and Skoog culture medium 702 for further culturing in step 208. The supplemented Murashige and Skoog culture medium 702 can comprise growth regulators configured to support the growth and development of nascent plant cells and coordinate intercellular communication. The supplemented Murashige and Skoog culture medium 702 will be discussed in more detail in the following sections.

FIG. 2B illustrates certain additional steps of the embodiment of the method 200 for producing substantially pathogen-free plants of the genus Cannabis. The method 200 can further comprise transferring a plantlet 704 grown from the meristematic tip 602 from the culturing plate 700 into a test tube 706 comprising additional supplemented Murashige and Skoog culture medium 702 after approximately 21 days to 30 days (or 3-4 weeks) in step 210. The method 200 can also comprise transferring the plantlet 704 from the test tube 706 into a large-tissue culture vessel 800 (see FIG. 8A) comprising Murashige and Skoog culture medium after approximately 28 days to 56 days (or 4-8 weeks) in step 212.

In addition, the method 200 can further comprise transferring the plantlet 704 growing in the large-tissue culture vessel 800 (see FIG. 8A) into a first rooting medium 804 (see FIG. 8B) after 28 days to 56 days to yield a young elite mother plant 802 in step 214. As will be discussed in more detail in the following sections, an “elite mother plant” can be a substantially pathogen-free mother plant produced from the methods described herein that can be used to create cloned plants or genetic copies (e.g., through cuttings). A “young elite mother plant” can be an immature or developing elite mother plant that is not yet ready to produce cuttings.

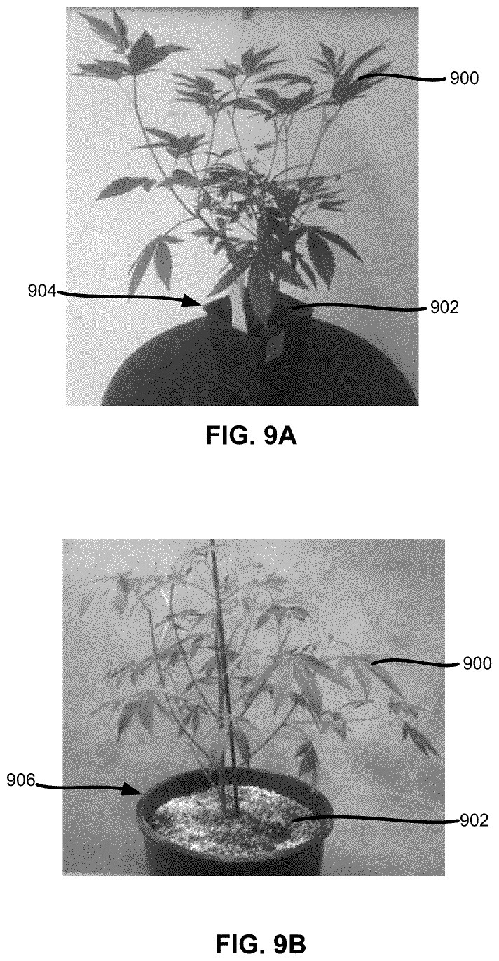

The method 200 can also comprise transferring the young elite mother plant 802 and at least a portion of the first rooting medium 804 into a second rooting medium 902 (see FIGS. 9A and 9B) after approximately 10 days to 16 days in the first rooting medium 804 in step 216. For example, the first rooting medium 804 can be a rock-wool cube and at least a portion of the rock-wool cube (or the entire rock-wool cube) can be transplanted into the second rooting medium 902. In some embodiments, the second rooting medium 902 can comprise soil, pumice, perlite, peat, coir, polymer stabilized rooting plugs, other types of mineral wool, or any combination thereof.

The method 200 can further comprise growing the young elite mother plant 802 in the second rooting medium 902 between approximately 7 days and 28 days to yield an elite mother plant 900 (see FIGS. 9A and 9B) in step 218. As will be discussed in more detail in the following sections, the elite mother plant 900 can be used to produce cuttings to produce cloned plants or genetic copies.

FIG. 3A is a black-and-white image of an embodiment of a heating chamber 300 used to heat the one or more progenitor plants 302. In some embodiments, the heating chamber 300 can be a temperature-controlled growth chamber designed for heating plant matter. For example, the heating chamber 300 can be a ThermoFisher Scientific™ Growth Chamber (Model No. 3768 or Catalog No. 846). In other embodiments, the heating chamber 300 can be any heating chamber having a temperature control thermostat and programmable heating element.

In some embodiments, the heating chamber 300 can have sliding glass doors for ease of access and viewing. Moreover, the heating chamber 300 can undertake a 24-hour heating cycle such that the heating element is constantly on during the heat treatment period.

In certain embodiments, the interior of the heating chamber 300 can comprise a number of fluorescent or incandescent lamps with adjustable lighting levels. Moreover, the heating chamber 300 can also comprise a number of fans to circulate air within the interior of the heating chamber 300.

In one embodiment, the heating element can be a heating strip positioned within an interior of the heating chamber 300. In other embodiments, the heating element can comprise heat lamps, heating fans, or a combination thereof.

In some embodiments, the progenitor plants 302 can be heated at a relative humidity of approximately 50%. In other embodiments, the progenitor plants 302 can be heated at a relative humidity of between approximately 50% and 60%. In additional embodiments, the progenitor plants 302 can be heated at a relative humidity of between approximately 60% and 70%. In alternative embodiments, the progenitor plants 302 can be heated at a relative humidity of between approximately 70% and 90%.

As previously discussed, the progenitor plants 302 can be plants infected by a pathogen. In one embodiment, the pathogen can be PCIA. In other embodiments, the progenitor plant 302 can be a plant infected by another pathogen such as a virus from the family Virgaviridae, a virus from the family Betaflexiviridae, a viroid from the family Pospiviroidae, a viroid from the family Avsunviroidae, a phytoplasma, or a combination thereof. For example, the progenitor plant 302 can be a plant infected by a pathogen such as a virus from the genus Tobamovirus, a virus from the genus Carlavirus, a viroid from any of the genera Pospiviroid, Hostuviroid, Cocadviroid, Apscaviroid, or Coleviroid, a parasitic bacteria from the genus Candidatus Phytoplasma, or a combination thereof.

In these and other embodiments, the progenitor plant 302 infected by the one or more pathogens can be exhibiting symptoms of infection or disease or be in an asymptomatic stage or phase. Given that certain Cannabis plants may be infected by PCIA other pathogens and exhibit little or no symptoms during the vegetative growth stage, the method 200 can also involve heating healthy plants that are suspected of being infected by PCIA or other pathogens. Such plants can be those cultivated near or in proximity to other symptomatic plants infected by PCIA or other pathogens.

FIG. 3B illustrates an embodiment of a heating schedule 304 undertaken by the heating chamber 300 to heat the one or more progenitor plants 302. The heating schedule 304 can comprise heating the one or more progenitor plants 302 at a first heating temperature 306 for a first heating duration 308, adjusting the heating temperature to a second heating temperature 310 and heating the one or more progenitor plants 302 at the second heating temperature 310 for a second heating duration 312, and repeating this alternating heating cycle until a total heating period 314 has elapsed.

In some embodiments, the first heating temperature 306 can be between approximately 95° F. (35° C.) and 104° F. (40° C.). For example, the first heating temperature 306 can be approximately 100° F. (37.78° C.). In these and other embodiments, the second heating temperature 310 can be between approximately 80° F. (26.67° C.) and 88° F. (31.11° C.). For example, the second heating temperature 310 can be approximately 85° F. (29.44° C.).

In some embodiments, the first heating duration 308 can be between approximately 3.75 hours to 4.25 hours. For example, the first heating duration 308 can be approximately 4.00 hours. In these and other embodiments, the second heating duration 312 can be approximately 3.75 hours to 4.25 hours. For example, the second heating duration 312 can be approximately 4.00 hours. In certain embodiments, the first heating duration 308 can be the same as the second heating duration 312. In alternative embodiments, the first heating duration 308 can differ from the second heating duration 312. As a more specific example, the heating schedule 304 can comprise heating the one or more progenitor plants 302 at alternating temperatures of approximately 100° F. and 85° F. where the progenitor plants 302 are heated at each temperature for approximately a four hour period for a total of 14 days.

In alternative embodiments not shown in FIG. 3B, the heating schedule 304 can comprise heating the one or more progenitor plants 302 at a constant heating temperature of between approximately 95° F. (35° C.) and 104° F. (40° C.). For example, the heating schedule 304 can comprise heating the one or more progenitor plants 302 at a constant heating temperature of approximately 100° F. (37.78° C.) for the total heating period 314.

In some embodiments, the total heating period 314 can be between approximately 13 days (approximately 312 hours) and 15 days (360 hours). For example, the total heating period 314 can be approximately 14 days (approximately 336 hours).

One unexpected discovery made by the applicants is that numerous cultivars of Cannabis sativa, Cannabis indica, and hybrids thereof can be heat treated using the heating schedule 304 disclosed herein without substantially impairing the viability of the meristematic tips 602 of such heat-treated plants 400 for further culturing. Moreover, the heating schedule 304 disclosed herein has been discovered to be optimal for the method 200 of producing substantially pathogen-free plants of the genus Cannabis or clones thereof.

FIG. 4 is a black-and-white image of embodiments of heat-treated plants 400. As shown in FIG. 4, the heated-treated plants 400 can be in a stressed state after undergoing heat treatment. One objective of subjecting the progenitor plants 302 to the heat treatment disclosed herein is to slow the progress of any infectious agents or pathogens which may have infected the progenitor plants 302. More specifically, one objective of subjecting the progenitor plants 302 to the heat treatment disclosed herein is to prevent the infectious agent or pathogen or byproducts produced thereby from reaching the shoot meristem of the heated progenitor plants.

As previously disclosed, the progenitor plants 302 can be in a vegetative growth stage when heated. For example, the progenitor plant 302 can be between the ages of 2 weeks to 3 weeks when heated. In other example embodiments, the progenitor plant 302 can be between the ages of 3 weeks to 4 weeks when heated. Such plants can have a height dimension of between approximately 6 inches (approximately 15.24 cm) and 18 inches (approximately 45.72 cm), as measured from the soil surface. One unexpected discovery made by the applicant is that progenitor plants 400 of the height dimension (between approximately 6 inches (approximately 15.24 cm) and 18 inches (approximately 45.72 cm)) disclosed herein are mostly able to withstand the heating schedule 304 disclosed herein without suffering irreparable harm to their meristematic tissue. Thus, Cannabis progenitor plants 302 of the height dimension disclosed herein are optimal for the heating step of the method 200 disclosed herein for producing substantially pathogen-free plants of the genus Cannabis or clones thereof.

FIG. 5A is a black-and-white image of a shoot segment 500 excised from the heat-treated plant 400. The shoot segment 500 can be a segment of a main stem of the heat-treated plant 400. The shoot segment 500 can also be a segment of a side shoot or branch of the plant. The shoot segment 500 can measure between approximately 3.0 inches (7.62 cm) and 5.0 inches (12.7 cm) in length. The shoot segment 500 can be excised using sterilized scalpels, shears, knives, or a combination thereof.

FIG. 5B illustrates an embodiment of a technique of surface sterilizing the excised shoot segment 500. In the embodiment shown in FIG. 5B, surface sterilizing the excised shoot segment 500 can comprise immersing the excised shoot segment 500 in a bleach solution 502. For example, a container 504 can be filled with the bleach solution 502 and the excised shoot segment 500 can be completely immersed in the bleach solution 502. In other embodiments, at least part of the excised shoot segment 500 such as the top of the shoot segment 500 is immersed in the bleach solution 502. The excised shoot segment 500 can be immersed in the bleach solution 502 for between approximately 10 minutes and 20 minutes. In one embodiment, the excised shoot segment 500 can be immersed in the bleach solution 502 for approximately 15 minutes. In alternative embodiments, the excised shoot segment 500 can be sprayed with the bleach solution 502 or the bleach solution 502 can be poured on the excised shoot segment 500.

The bleach solution 502 can comprise approximately 2.475% (w/v %) of sodium hypochlorite. The bleach solution 520 can be made by diluting a bleach solution (e.g., HDX™ Germicidal Bleach or Clorox™ Germicidal Bleach) comprising approximately 8.25% (w/v %) of sodium hypochlorite (which yield 7.86% of available chlorine) with distilled water. For example, the bleach solution 502 can be made by combining approximately 30.0% (v/v %) germicidal bleach solution comprising 8.25% sodium hypochlorite with approximately 70.0% (v/v %) distilled water. The end result is a bleach solution 502 comprising approximately 2.475% (w/v %) of sodium hypochlorite.

FIG. 6 illustrates the anatomy of a distal portion 600 of a shoot segment of a plant of the genus Cannabis. FIG. 6 illustrates a location of the meristematic tip 602 relative to the rest of the distal portion 600 of the shoot segment. The method 200 can comprise excising the meristematic tip 602 using a sterilized scalpel under microscopy. The meristematic tip 602 can refer to an apical portion of the shoot segment equal to approximately 0.50 mm in length. In other embodiments, the meristematic tip 602 can refer to an apical portion of the shoot segment less than approximately 0.50 mm in length such as between 0.30 mm and 0.50 mm. In alternative embodiments, the meristematic tip 602 can refer to an apical portion of the shoot segment greater than 0.50 mm in length such as between approximately 0.55 mm and 0.75 mm.

In some embodiments, the meristematic tip 602 can comprise the apical dome and a limited number of young leaf primordia. The meristematic tip 602 excludes any differentiated provascular tissues or vascular tissues. For example, care should be taken not to excise any part of the shoot segment comprising the procambium 604, xylem 606, or phloem 608.

The objective of excising the meristematic tip 602 is to excise meristem tissue without excising any part of the vasculature of the plant that may comprise viruses, viroids, or other pathogens. One advantage of heat treating the plant prior to excision is that the heat treatment slows the progress of any viral or other pathogen infections and ideally prevents the pathogen from reaching the shoot apical meristem of the plant.

The method 200 can comprise transferring the excised meristematic tips 602 (also referred to as the meristem explants) into culturing plates 700 (see FIG. 7A) comprising supplemented Murashige and Skoog culture medium 702. When initially placed on the culturing plates 700, the meristematic tips 602 can be barely perceptible to the naked eye.

FIG. 7A is a black-and-white image of meristematic tips 602 that have been growing on culturing plates 700 comprising supplemented Murashige and Skoog culture medium 702 for approximately two weeks. In some embodiments, the culturing plates 700 can be transparent Petri dishes such as borosilicate or polyethylene Petri dishes. In these and other embodiments, the culturing plates 700 can be a substantially circular Petri dish having an outer diameter of between approximately 2.75 inches (approximately 70.0 mm) and 3.93 inches (approximately 100 mm) and a plate depth of approximately 0.6 inches (approximately 15 mm). In alternative embodiments, the culturing plates 700 can be compartmentalized or be substantially rectangular in shape.

As shown in FIG. 7A, multiple meristematic tips 602 can be cultured within the same culturing plate 700. Moreover, as shown in FIG. 7A, the culturing plates 700 can be covered by plastic wrap, cling wrap, or a combination thereof to prevent contamination and moisture loss.

The supplemented Murashige and Skoog culture medium 702 can comprise Murashige and Skoog culture medium supplemented with plant growth-regulators. Murashige and Skoog culture medium is plant culture medium having ingredients similar to those presented in Murashige, Toshio, and Folke Skoog. “A revised medium for rapid growth and bio assays with tobacco tissue cultures.” Physiologia Plantarum 15.3 (1962): 473-497. Commercially available Murashige and Skoog culture medium can include those distributed by ThermoFisher Scientific Inc., Sigma-Aldrich Co. LLC, or W.W. Grainger, Inc.

The supplemented Murashige and Skoog culture medium 702 can comprise plant growth-regulators including benzyladenine (6-benzylaminopurine), naphthaleneacetic acid, gibberellic acid, or a combination thereof. In one embodiment, the supplemented Murashige and Skoog culture medium 702 can comprise plant growth-regulators in the amount of 1.0 mg/L of benzyladenine (6-benzylaminopurine), 0.1 mg/L of naphthaleneacetic acid (1-naphthaleneacetic acid), and 0.1 mg/L of gibberellic acid. The growth regulators can be configured to support the growth and development of nascent plant cells and coordinate intercellular communication.

FIG. 7B is a black-and white image of a plantlet 704 grown from a meristematic tip 602 within a test tube 706 comprising supplemented Murashige and Skoog culture medium 702. The method 200 can comprise transferring a plantlet 704 grown from the meristematic tip 602 from the culturing plate 700 into the test tube 706 after approximately 21 to 30 days.

In some embodiments, the test tube 706 can be a plastic test tube made in part of polystyrene, polyethylene, or a combination thereof. In other embodiments, the test tube 706 can be a glass test tube made in part of borosilicate glass (e.g., Pyrex™ test tubes). In some embodiments, the test tube 706 can have a tube diameter of between approximately 0.98 inches (25.0 mm) and 1.50 inches (38.0 mm). The test tube 706 can be filled partially with supplemented Murashige and Skoog culture medium 702 but have plenty of room for the plantlet to grow within the test tube 706.

FIG. 8A is a black-and-white image of three types of culturing vessels used as part of the method 200 of producing substantially pathogen-free plants including culturing plates 700, test tubes 706, and large tissue-culture vessels 800. The method 200 can further comprise transferring the plantlet 704 from a test tube 706 into a large-tissue culture vessel 800 comprising Murashige and Skoog culture medium 702 after approximately 28 days to 56 days.

In some embodiments, the large tissue-culture vessels 800 can be substantially cylindrical in shape. In other embodiments, the large tissue-culture vessels 800 can be substantially frustoconic in shape. For example, the large tissue-culture vessels 800 can have a carrying capacity of between approximately 350 mL and 500 mL. When the large tissue-culture vessel 800 is substantially shaped as an upside-down frustoconic, the tissue-culture vessel 800 can have a top diameter of approximately 4.625 inches (approximately 11.75 cm) and a bottom diameter of approximately 3.375 inches (approximately 8.57 cm).

In some embodiments, the large tissue-culture vessels 800 can be made in part from polyethylene, polypropylene, or a combination thereof. In other embodiments, the large tissue-culture vessels 800 can be made in part from borosilicate glass.

FIG. 8B is a black-and-white image of young elite mother plants 802 grown from cultured meristematic tips 602 excised from heat-treated plants 400 and acclimated for ambient ex-vitro growing conditions. As shown in FIG. 8B, the young elite mother plants 802 are rooted in a first rooting medium 804. The young elite mother plants 802 are developed from the plantlets 704 grown in the large tissue-culture vessels 800 shown in FIG. 8A.

As previously discussed, the plantlets 704 can be transferred from the large-tissue culture vessels 800 (see FIG. 8A) into the first rooting medium 804 as part of the method 200. The 704 can be transferred from the large-tissue culture vessels 800 (see FIG. 8A) into the first rooting medium 804 after approximately 28 days to 56 days spent in the large-tissue culture vessels 800. Once the plantlets 704 have taken root in the first rooting medium 804, the plantlets 704 can be considered young elite mother plants 802.

In some embodiments, the first rooting medium 804 can be rock-wool (e.g., a rock-wool cube) or other types of mineral wool. In these and other embodiments, the first rooting medium 804 can also comprise soil, pumice, perlite, peat, coir, polymer stabilized rooting plugs, or any combination thereof.

For purposes of this disclosure, an “elite mother plant” can be a substantially pathogen-free mother plant of the genus Cannabis produced from the methods and treatment steps described herein. Elite mother plants can be used to create cloned plants or genetic copies through cuttings and other propagation methods. A “young elite mother plant” can be an immature or developing elite mother plant that is not yet ready for cloning or propagation.

The method 200 can also comprise transferring the young elite mother plant 802 and at least a portion of the first rooting medium 804 into a second rooting medium 902 (see FIGS. 9A and 9B) after approximately 10 days to 16 days in the first rooting medium 804. For example, the first rooting medium 804 can be a rock-wool cube and at least a portion of the rock-wool cube (or the entire rock-wool cube) can be transplanted along with the young elite mother plant 802 into the second rooting medium 902. In one embodiment, the second rooting medium 902 can be soil. In this and other embodiments, the second rooting medium 902 can also comprise pumice, perlite, peat, coir, polymer stabilized rooting plugs, other types of mineral wool, or any combination thereof.

The method 200 can further comprise growing the young elite mother plants 802 in the second rooting medium 902 to yield an elite mother plant 900 (see FIGS. 9A and 9B). The young elite mother plants 802 can be allowed to grow in the second rooting medium 902 for between approximately 7 days and 28 days. As will be discussed in more detail in the following sections, the elite mother plant 900 can be harvested for cuttings to produce cloned plants or genetic copies.

One unexpected discovery made by the applicants is that elite mother plants produced, in part, by the method 200 disclosed herein yielded, on a population level, a greater percentage of phenotypically healthier propagates or clones than mother plants produced by other methods. Another unexpected discovery made by the applicants is that elite mother plants produced, in part, by the method 200 disclosed herein produced, on average, more cannabinoid or terpenoid-rich plant matter than mother plants produced by other methods.

FIG. 9A is a black-and-white image of a miniature-sized elite mother plant 900 (also referred to as a mini-elite mother plant). A miniature-sized elite mother plant 900 can be an elite mother plant 900 grown in a small container 904. For example, the small container 904 can be an upside-down truncated square pyramidal container having a top length and top width dimension of approximately 3.5 inches (or approximately 8.89 cm), a height dimension of approximately 3.375 inches (or approximately 8.57 cm), and a bottom length and width dimension of approximately 2.5 inches (or approximately 6.35 cm). In other embodiments, the small container 904 can be a cylindrical or frustoconic container having a top diameter of approximately 3.5 inches (or approximately 8.89 cm). In additional embodiments, the miniature-sized elite mother plants 900 can be grown in a substantially cuboidal container having a length, height, and width dimension of between approximately 3.0 inches (or approximately 7.62 cm) and 5.0 inches (or 12.7 cm).

From the time the young elite mother plants 802 are transplanted into the small containers 904, the young elite mother plants 802 can be allowed to grow in the small containers 904 for between approximately 7 days and 21 days (or 1 week to 3 weeks) until the branches of such plants are long enough to be harvested for cuttings.

FIG. 9B is a black-and-white image of a regular-sized elite mother plant 900. A regular-sized elite mother plant 900 can be an elite mother plant 900 grown in a large container 906. For example, the regular-sized elite mother plant 900 can be grown in a container or pot having a container volume of between approximately 5 gallons (approximately 18.9 liters) and 7 gallons (approximately 26.5 liters). From the time the young elite mother plants 802 are transplanted into the large containers 906, the young elite mother plants 802 can be allowed to grow in the large containers 906 for between approximately 14 days and 28 days (or 2 weeks to 4 weeks) until the branches of such plants are long enough to be harvested for cuttings.

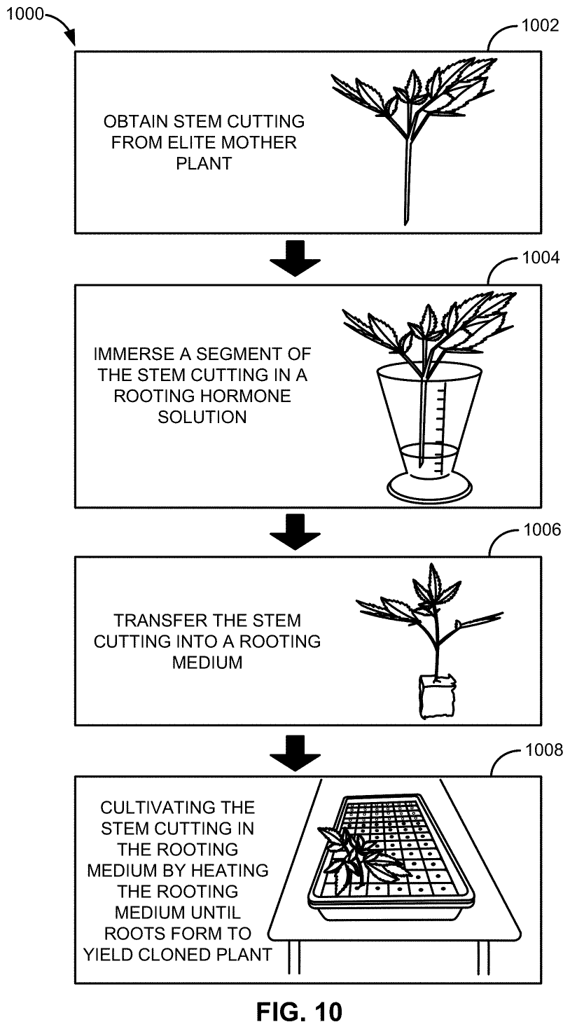

FIG. 10 illustrates certain steps of a method 1000 for producing cloned plants of the genus Cannabis from substantially pathogen-free elite mother plants 900. The elite mother plants 900 can be produced from the method 200 disclosed herein. The steps of method 1000 can be considered additional steps of method 200 or a continuation of method 200.

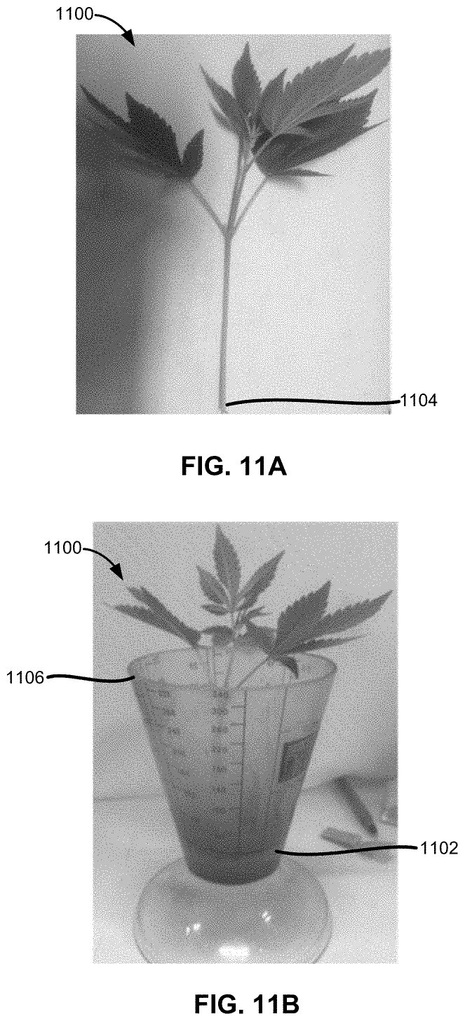

The method 1000 can comprise obtaining a stem cutting 1100 (see FIGS. 11A and 11B) of the elite mother plant 900 in step 1002. The stem cutting 1100 can be obtained by cutting a main stem or a branch of the elite mother plant 900 using sterilized cutting instruments. In some embodiments, the cutting instruments can comprise pruning shears, scissors, scalpels, or a combination thereof. The stem cutting 1100 can measure between approximately 3.0 inches (7.62 cm) and 7.0 inches (17.78 cm) in length.

The method 1000 can also comprise immersing at least a segment of the stem cutting 1100 in a rooting hormone solution 1102 (see FIG. 11B) in step 1004. In one embodiment, the excised or cut end 1104 (see FIG. 11A) of the stem cutting 1100 can be immersed or dipped in the rooting hormone solution 1102 for between approximately 5 seconds and 10 seconds. For example, the rooting hormone solution 1102 can be poured into a solution container 1106 (e.g., a beaker or cup) and the excised or cut end 1004 of the stem cutting 1100 can be dipped or immersed in the rooting hormone solution 1102 for between approximately 5 seconds and 10 seconds.

In some embodiments, the rooting hormone solution 1102 can comprise indole-3-butyric acid (IBA) and 1-napthaleneacetic acid as active ingredients. For example, the rooting hormone solution 1102 can be a diluted solution comprising a concentrated rooting hormone solution. The concentrated rooting hormone solution can comprise approximately 1.0% (w/v %) IBA and 0.5% (w/v %) 1-napthaleneacetic acid as active ingredients. The rooting hormone solution 1102 can also comprise ethanol and isopropyl alcohol. As a more specific example, the rooting hormone solution 1102 can be a diluted solution comprising 10% (v/v %) of Dip 'N Grow® Liquid Rooting Concentrate distributed by Dip 'N Grow Inc. In other embodiments, the rooting hormone solution 1102 can be other rooting hormones in the form of powders or gels added to an aqueous solution.

The method 1000 can further comprise transferring the stem cutting 1100 into a temperature-controlled rooting medium 1200 after dipping or immersing the segment of the stem cutting 1100 in the rooting hormone solution 1102 in step 1006. In one embodiment, the temperature-controlled rooting medium 1200 can be a heated rock-wool rooting medium.

The method 1000 can also comprise further cultivating the stem cutting 1100 in the temperature-controlled rooting medium 1200 until roots form to yield a cloned plant in step 1008. In one embodiment, further cultivating the stem cutting 1100 in the temperature-controlled rooting medium 1200 can comprise heating the rooting medium to a temperature of approximately 80° F. (26.67° C.) using a heating system or heating device and maintaining the temperature of the rooting medium using the heating system or heating device. Moreover, step 1008 can also comprise maintaining a relative humidity of 60% using a fog generating machine or fogger and maintaining a relative ambient temperature of approximately 70° F. The stem cutting 1100 can be further cultivated in this manner until the stem cutting 1100 takes root in approximately 10 days to 16 days. Once the stem cutting 1100 has taken root in the rooting medium, the rooted plant can be considered a cloned plant or clone of the elite mother plant 900.

One unexpected discovery made by the applicants is that cloned plants produced, in part, by a combination of the method 200 and method 900 disclosed herein were, on a population level, healthier than cloned plants produced by other methods. Another unexpected discovery made by the applicants is that cloned plants produced by the methods disclosed herein produced, on average, more cannabinoid or terpenoid-rich plant matter than cloned plants produced by other methods.

FIG. 11A is a black-and-white image of a stem cutting 1100 obtained from an elite mother plant 900. As previously discussed, the stem cutting 1100 can be obtained by cutting a main stem or a branch of the elite mother plant 900 using sterilized cutting instruments. In some embodiments, the cutting instruments can comprise pruning shears, scissors, scalpels, or a combination thereof. The stem cutting 1100 can measure between approximately 3.0 inches (7.62 cm) and 7.0 inches (17.78 cm) in length. As shown in FIG. 11A, the stem cutting 1100 can comprise one or more leaves, nodes, internodes, and branches. Moreover, FIG. 11A shows that the stem cutting 1100 can have an excised or cut end 1104.

FIG. 11B is a black-and-white image of a segment of the stem cutting 1100 immersed in a rooting hormone solution 1102. For example, the excised or cut end 1104 along with a segment of the stem cutting 1100 between approximately 0.5 inches (1.27 cm) and 1.0 inches (2.54 cm) in proximity to the cut end 1104 can be immersed or dipped into the rooting hormone solution 1102. The segment of the stem cutting 1100 can be immersed or dipped in the rooting hormone solution 1102 for between approximately 5 seconds and 10 seconds.

As previously discussed, in some embodiments, the rooting hormone solution 1102 can comprise indole-3-butyric acid (IBA) and 1-napthaleneacetic acid as active ingredients. For example, the rooting hormone solution 1102 can be a diluted solution comprising a concentrated rooting hormone solution. The concentrated rooting hormone solution can comprise approximately 1.0% (w/v %) IBA and 0.5% (w/v %) 1-napthaleneacetic acid as active ingredients. The rooting hormone solution 1102 can also comprise ethanol and isopropyl alcohol. As a more specific example, the rooting hormone solution 1102 can be a diluted solution comprising 10% (v/v %) of Dip 'N Grow® Liquid Rooting Concentrate distributed by Dip 'N Grow Inc. In other embodiments, the rooting hormone solution 1102 can be other rooting hormones in the form of powders or gels added to an aqueous solution.

The rooting hormone solution 1102 can be poured into a solution container 1106 such as a measuring cup or beaker and the segment of the stem cutting 1100 in proximity to the excised or cut end 1004 can be immersed in the rooting hormone solution 1102 for between approximately 5 seconds and 10 seconds. In some embodiments, the solution container 1106 can be a polymeric container. In other embodiments, the solution container 1106 can be a ceramic or glass container, a stainless steel container, or a combination thereof.

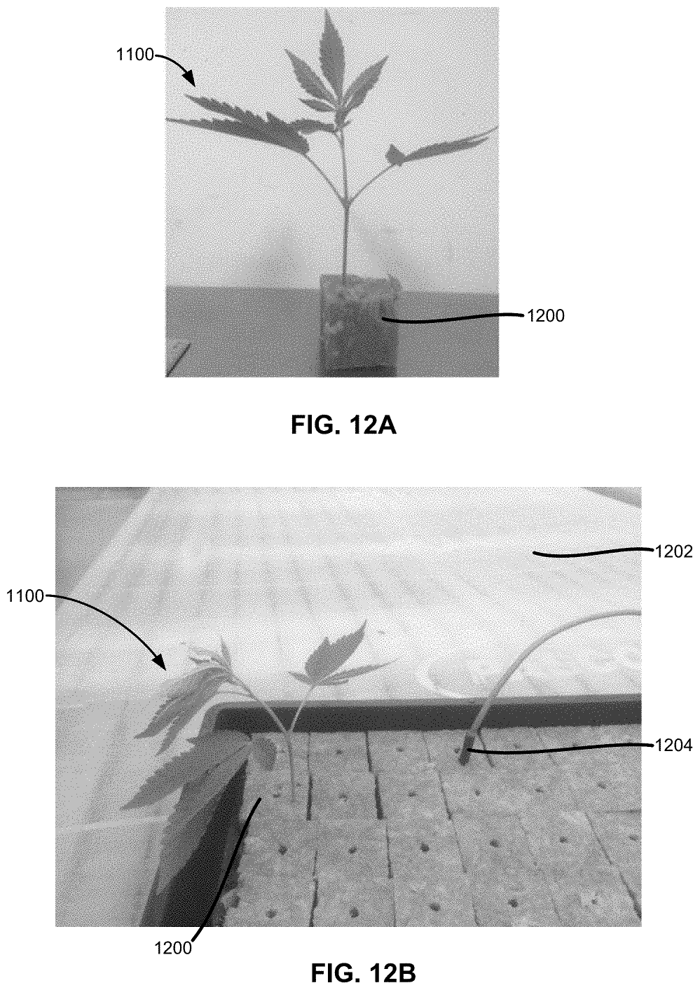

FIG. 12A is a black-and-white image of the stem cutting 1100 placed in a rooting medium 1200. In one embodiment, the rooting medium 1200 can be rock-wool rooting medium. In this and other embodiments, the rooting medium 1200 can be or comprise a polymeric rooting medium, soil, pumice, perlite, peat, coir, polymer stabilized rooting plugs, other types of mineral wool, or any combination thereof. The immersed or dipped segment of the stem cutting 1100 can be inserted or otherwise placed in the rooting medium 1200 soon after being removed from the rooting hormone solution 1102.

FIG. 12B is a black-and-white image showing the stem cutting 1100 being further cultivated in a temperature-controlled rooting medium 1200 to yield a cloned plant. The rooting medium 1200 can be heated to a temperature of approximately 80° F. (26.67° C.) using a heating mat or a hydronic heating system. For example, the heating mat and hydronic heating system can each comprise a heating surface 1202 (e.g., a mat surface, heated table top or bench top surface, etc.) and the stem cuttings 1100 in rooting media 1200 can be placed on top of the heating surface 1202 or in a tray or pan on top of the heating surface 1202 (i.e., the rooting medium 1200 can be bottom heated). A temperature probe or sensor 1204 can also be placed in contact with the rooting medium 1200 to gauge or monitor the temperature of the rooting medium 1200. The temperature probe or sensor 1204 can be electrically coupled to a programmable controller which can also control a heat-generating apparatus used to heat the heating surface 1202. The programmable controller can turn the heat-generating apparatus on or off in order to heat or cool down the heating surface 1202 such that the temperature of the rooting medium 1200 (as detected by the temperature probe or sensor 1204) is maintained close to a predetermined set point (e.g., approximately 80° F. or 26.67° C.).

For large scale cloning operations, rooting media 1200 comprising stem cuttings 1100 can be heated using a hydronic heating system. In one embodiment, the hydronic heating system can be an under-bench heating system distributed by BioTherm, Inc. For small scale cloning operations, rooting media 1200 comprising stem cuttings 1100 can be heated using an electrical heating mat. In one embodiment, the heating mat can be a Jump Start™ Seedling Heating Mat distributed by Hydrofarm, Inc.

Cultivating the stem cutting 1100 can also comprise maintaining a relative humidity of 60% using a fog generating machine or fogger (e.g., a FOGCO™ Revolution Humidification Fan distributed by Fogco Systems, Inc.) and maintaining a relative ambient temperature of approximately 70° F. The stem cutting 1100 can be cultivated in this manner until the stem cutting 1100 takes root in approximately 10 days to 16 days. Once the stem cutting 1100 has taken root in the rooting medium 1200, the rooted plant can be considered a cloned plant or clone of the elite mother plant 900.

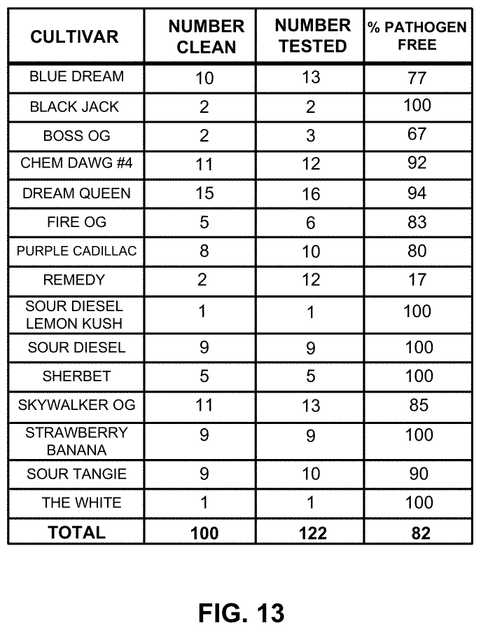

FIG. 13 is a table showing the results of tests conducted in April 2018 on plants of the genus Cannabis produced by the methods described herein for incidence of pathogen infections. As shown in FIG. 13, a total of 122 plants of various cultivars were tested for PCIA and approximately 82% of the plants tested were pathogen free. Moreover, when testing data for cultivar Remedy is omitted, the percentage increases to approximately 89%. Since previous plant epidemiological studies have estimated the incidence of PCIA to be as high as approximately 35% (and possibly much higher when infected asymptomatic plants are taken into account) in untreated cannabis nursery stock plants, a decrease of such incidence to approximately 18% (or 11% when outlier data for cultivars such as Remedy is removed) is a significant advance in the field of cannabis cultivation. Moreover, the methods described herein can involve testing the plants produced by the process steps disclosed herein (for example, the young plants 802 shown in FIG. 8B) for PCIA and culling plants that test positive for PCIA. By doing so, the clones created from the elite mother plants which have survived the culling process will be nearly 100% pathogen free.

As shown in FIG. 13, one unexpected discovery made by the applicants is that the methods disclosed herein are effective for a wide range of Cannabis sativa plants including numerous commercially-valuable cultivars or strains. Moreover, another unexpected discovery made by the applicants is that certain cultivars responded exceptionally well to the methods disclosed herein. For example, plants of the Dream Queen cultivar or strain, the Sour Diesel cultivar or strain, the Sherbet cultivar or strain, and the Strawberry Banana cultivar or strain responded better than other Cannabis sativa cultivars. More testing will need to be done to further corroborate the efficacy of the methods disclosed herein.

A number of embodiments have been described. Nevertheless, it will be understood by one of ordinary skill in the art that various modifications may be made without departing from the spirit and scope of the embodiments. In addition, the flowcharts or logic flows depicted in the figures do not require the particular order shown, or sequential order, to achieve desirable results. In addition, other steps or operations may be provided, or steps or operations may be eliminated, from the described flows, and other components may be added to, or removed from, the described systems. Accordingly, other embodiments are within the scope of the following claims.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art to which this invention belongs.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure. Accordingly, the specification and/or drawings may be regarded in an illustrative rather than a restrictive sense.

1. A method of producing plants of the genus Cannabis, the method comprising:
heating a progenitor plant of the genus Cannabis with a height dimension of between 6 inches and 18 inches as measured from a soil surface within a heating chamber resulting in a heat-treated plant, wherein the progenitor plant is in a vegetative growth stage when heated;
surface sterilizing a shoot segment of the heat-treated plant with a bleach solution;
excising a shoot apical meristematic tip of the shoot segment of the heat-treated plant; and
transferring the shoot apical meristematic tip of the shoot segment of the heat-treated plant into a culturing plate comprising a supplemented Murashige and Skoog culture medium for further culturing of the shoot apical meristematic tip, wherein the supplemented Murashige and Skoog culture medium comprises 1.0 mg/L of benzyladenine, 0.1 mg/L of naphthaleneacetic acid, and 0.1 mg/L of gibberellic acid.

2. The method of claim 1, wherein heating the progenitor plant comprises heating the progenitor plant at a constant temperature of between approximately 95° F. and 104° F., wherein the progenitor plant is heated at the constant temperature for a total of 14 days.

3. (canceled)

4. The method of claim 1, wherein excising the shoot apical meristematic tip of the shoot segment comprises excising a distal apical portion of the shoot segment equal to or less than approximately 0.5 mm in size, wherein the distal apical portion of the shoot segment comprises meristem tissue.

5. (canceled)

6. The method of claim 1, wherein surface sterilizing the shoot segment of the heat-treated plant comprises immersing the shoot segment in the bleach solution for between approximately 10 minutes and 20 minutes.

7. The method of claim 6, wherein the bleach solution comprises approximately 2.475% (w/v %) of sodium hypochlorite.

8. The method of claim 1, further comprising transferring a plantlet grown from the shoot apical meristematic tip from the culturing plate into a test tube comprising the supplemented Murashige and Skoog culture medium after 21 days to 30 days.

9. The method of claim 8, further comprising transferring the plantlet growing in the test tube into a tissue culture vessel comprising Murashige and Skoog culture medium after 28 days to 56 days, wherein the tissue culture vessel has a carrying capacity greater than the test tube.

10. The method of claim 9, further comprising:
transferring the plantlet growing in the tissue culture vessel into a first rooting medium after 28 days to 56 days to yield a young elite mother plant;
transferring the young elite mother plant and at least a portion of the first rooting medium into a second rooting medium after 10 days to 16 days; and
growing the young elite mother plant in the second rooting medium between 7 days and 28 days to yield an elite mother plant.

11.-30. (canceled)

31. A method of producing a viroid-free plant of the genus Cannabis from a progenitor plant infected by a viroid, the method comprising:
heating the progenitor plant of the genus Cannabis infected by the viroid within a heating chamber resulting in a heat-treated plant, wherein the progenitor plant is in a vegetative growth stage when heated, wherein a height dimension of the progenitor plant heated in the heating chamber is between 6 inches and 18 inches as measured from a soil surface;
surface sterilizing a shoot segment of the heat-treated plant with a bleach solution;
excising a shoot apical meristematic tip of the shoot segment; and
transferring the shoot apical meristematic tip into a culturing plate comprising a supplemented Murashige and Skoog culture medium for further culturing of the shoot apical meristematic tip, wherein the supplemented Murashige and Skoog culture medium comprises 1.0 mg/L of benzyladenine, 0.1 mg/L of naphthaleneacetic acid, and 0.1 mg/L of gibberellic acid.

32. The method of claim 31, further comprising:
transferring a plantlet grown from the shoot apical meristematic tip from the culturing plate into a test tube comprising the supplemented Murashige and Skoog culture medium after 21 days to 30 days;
transferring the plantlet growing in the test tube into a tissue culture vessel comprising Murashige and Skoog culture medium after 28 days to 56 days, wherein the tissue culture vessel has a carrying capacity greater than the test tube;
transferring the plantlet growing in the tissue culture vessel into a first rooting medium after 28 days to 56 days to yield a young elite mother plant;
transferring the young elite mother plant and at least a portion of the first rooting medium into a second rooting medium after 10 days to 16 days; and
growing the young elite mother plant in the second rooting medium between 7 days and 28 days to yield the elite mother plant.

33. The method of claim 31, wherein heating the progenitor plant comprises heating the progenitor plant at a constant temperature of between approximately 95° F. and 104° F., wherein the progenitor plant is heated at the constant temperature for a total of 14 days.

34. The method of claim 31, wherein excising the shoot apical meristematic tip of the shoot segment comprises excising a distal apical portion of the shoot segment equal to or less than approximately 0.5 mm in size, wherein the distal apical portion of the shoot segment comprises meristem tissue.

35. The method of claim 31, wherein surface sterilizing the shoot segment of the heat-treated plant comprises immersing the shoot segment in the bleach solution for between approximately 10 minutes and 20 minutes, wherein the shoot apical meristematic tip is excised from the shoot segment previously immersed in the bleach solution for between approximately 10 minutes and 20 minutes.

36. The method of claim 35, wherein the bleach solution comprises approximately 2.475% (w/v %) of sodium hypochlorite.

37. A method of producing an elite mother plant of the genus Cannabis from a progenitor plant infected by Putative Cannabis Infectious Agent (PCIA), the method comprising:
heating the progenitor plant of the genus Cannabis infected by PCIA within a heating chamber resulting in a heat-treated plant, wherein the progenitor plant is in a vegetative growth stage when heated, wherein a height dimension of the progenitor plant heated in the heating chamber is between 6 inches and 18 inches as measured from a soil surface;
surface sterilizing a shoot segment of the heat-treated plant with a bleach solution;
excising a shoot apical meristematic tip of the shoot segment;
transferring the shoot apical meristematic tip into a culturing plate comprising a supplemented Murashige and Skoog culture medium for further culturing of the shoot apical meristematic tip, wherein the supplemented Murashige and Skoog culture medium comprises 1.0 mg/L of benzyladenine, 0.1 mg/L of naphthaleneacetic acid, and 0.1 mg/L of gibberellic acid;
transferring a plantlet grown from the shoot apical meristematic tip from the culturing plate into a test tube comprising the supplemented Murashige and Skoog culture medium after 21 days to 30 days;
transferring the plantlet growing in the test tube into a tissue culture vessel comprising Murashige and Skoog culture medium after 28 days to 56 days, wherein the tissue culture vessel has a carrying capacity greater than the test tube;
transferring the plantlet growing in the tissue culture vessel into a first rooting medium after 28 days to 56 days to yield a young elite mother plant;
transferring the young elite mother plant and at least a portion of the first rooting medium into a second rooting medium after 10 days to 16 days; and
growing the young elite mother plant in the second rooting medium between 7 days and 28 days to yield the elite mother plant.

38. The method of claim 37, wherein heating the progenitor plant comprises heating the progenitor plant at a constant temperature of between approximately 95° F. and 104° F., wherein the progenitor plant is heated at the constant temperature for a total of 14 days.

39. The method of claim 37, wherein excising the shoot apical meristematic tip of the shoot segment comprises excising a distal apical portion of the shoot segment equal to or less than approximately 0.5 mm in size, wherein the distal apical portion of the shoot segment comprises meristem tissue.

40. The method of claim 37, wherein surface sterilizing the shoot segment of the heat-treated plant comprises immersing the shoot segment in the bleach solution for between approximately 10 minutes and 20 minutes, wherein the shoot apical meristematic tip is excised from the shoot segment previously immersed in the bleach solution for between approximately 10 minutes and 20 minutes.

41. The method of claim 40, wherein the bleach solution comprises approximately 2.475% (w/v %) of sodium hypochlorite.
